(19) United States
(12) Patent Application Publication
(10) Pub. No.: US 2018/0056031 A1
(43) Pub. Date: Mar. 1, 2018

(54) Split Tip Catheter

(71) Applicant: VascuTech Medical LLC, Oaks, PA (US)

(72) Inventor: Mahase Nardeo, Oaks, PA (US)

(73) Assignee: VascuTech Medical LLC, Oaks, PA (US)

(21) Appl. No.: 15/680,496

(22) Filed: Aug. 18, 2017

Related U.S. Application Data
(60) Provisional application No. 62/379,341, filed on Aug. 25, 2016.

(51) Int. Cl. A61M 25/00; A61M 1/36

(52) U.S. Cl. CPC A61M 25/003; A61M 25/0068; A61M 2025/0034; A61M 25/0069; A61M 1/3661

(57) ABSTRACT

The present disclosure relates to catheter assembly and components thereof. In one embodiment, a catheter assembly includes a catheter including a body with a proximal end and a distal end, wherein the body includes a first lumen and a second lumen. The lumens are substantially symmetrical and are joined together and split at the distal ends thereof. The distal ends are bent apart from each other such that the catheter resembles a Y-shape when viewed from the front. Each of the distal ends comprises a U-shaped cutout to prevent the inflow of blood from becoming blocked. Because the lumens are substantially equal in size, shape, and dimension, the connection can be reversed without high recirculation if one lumen is blocked.

### CROSS-REFERENCE TO RELATED APPLICATIONS

This application claims the benefit of U.S. Provisional Patent Application No. 62/379,341, filed Aug. 25, 2016, which is hereby incorporated by reference in its entirety.

### FIELD OF THE INVENTION

The present invention relates generally to medical devices, namely, catheters. More particularly, the present invention is directed to a split tip catheter with a reversible connection.

### BACKGROUND OF THE INVENTION

Split tip catheters are used widely in dialysis. As the name implies, split tip catheters comprise two lumens having a split tip on one end thereof. Conventional split tip catheters comprise staggered tips to prevent blood recirculation. Blood recirculation occurs when dialyzed blood exiting the outflow lumen directly reenters the inflow lumen, thereby bypassing the systemic circulation. Recirculation reduces the effectiveness of dialysis by diluting the inflow concentration and reducing diffusion across the dialyzer membrane.

While existing split tip catheters reduce blood recirculation, the staggered construction designates a venous lumen and an arterial lumen. Thus, if one lumen is blocked, then the entire catheter assembly must be replaced. Additionally, the tips of existing catheters can block the intake of blood if one or both of the tips are positioned against the blood vessels of a patient. In this regard, the invention described herein addresses these problems.

### SUMMARY OF THE INVENTION

The following discloses a simplified summary of the specification in order to provide a basic understanding of some aspects of the specification. This summary is not an extensive overview of the specification. It is intended to neither identify key or critical elements of the specification nor delineate the scope of the specification. Its sole purpose is to disclose some concepts of the specification in a simplified form as a prelude to the more detailed description that is disclosed later.

In various embodiments, the present invention comprises a substantially symmetrical catheter assembly comprising a first lumen and a second lumen, wherein the two lumens are joined together over the length thereof but are split at the distal ends thereof. The lumens can be split at the distal ends using a heated die, wherein the lumens are separated via a septum that is disposed in the middle of the die. The distal ends are bent apart from each other such that the catheter resembles a Y-shape when viewed from the front. In various embodiments, the lumens are inserted into individual mandrel bends that are pre-shaped. The lumens are then heated to thermoset into the Y-shape.

Additionally, each of the distal ends comprises a U-shaped cutout to prevent the inflow of blood from becoming blocked. The U-shaped cutouts can be formed via a heated die. Because the lumens are not specifically designated as a venous lumen or an arterial lumen, the connection can be reversed without high recirculation if one lumen is blocked.

### BRIEF DESCRIPTION OF THE DRAWINGS

FIG. 1 shows a close-up view of an exemplary embodiment of a distal end of one of the lumens of the split tip catheter of the present invention.

FIG. 2 shows an overhead view of the split tip catheter of the present invention.

FIG. 3 shows a close-up view of the distal end of the split tip catheter of the present invention.

FIG. 4 shows a cross sectional view of the proximal end of the split tip catheter of the present invention.

### DETAILED DESCRIPTION OF THE INVENTION

For purposes of promoting an understanding of the present disclosure, reference is made to the embodiments illustrated in the above-referenced drawings. The following detailed description of the exemplary embodiments will make clear the arrangement, size relationships, and manner of using the components shown herein.

In the following discussion, the terms “proximal” and “distal” are used to describe the axial ends of the catheter, as well as the axial ends of various component features. The “proximal” end is used in a conventional manner to refer to the end of the catheter (or component) that is closest to the operator during use of the assembly. The “distal” end is used in a conventional manner to refer to the end of the catheter (or component) that is initially inserted into the patient, or that is closest to the patient. Additionally, those skilled in the art will appreciate that the catheter assembly described herein is suitable for multiple uses involving inflow and outflow of body fluids from a body vessel of a patient.

Moreover, the word “exemplary” is used herein to mean serving as an example, instance, or illustration. Any aspect or design described herein as “exemplary” is not necessarily to be construed as preferred or advantageous over other aspects or designs. Rather, use of the word exemplary is intended to disclose concepts in a concrete fashion. As used in this application, the term “or” is intended to mean an inclusive “or” rather than an exclusive “or.”

Additionally, the articles “a” and “an” as used in this application and the appended claims should generally be construed to mean “one or more” or “at least one” unless specified otherwise or clear from context to be directed to a singular form. Similarly, the terms “plurality” and “a plurality” as used herein includes, for example, “multiple” or “two or more.” For example, “a plurality of items” includes two or more items.

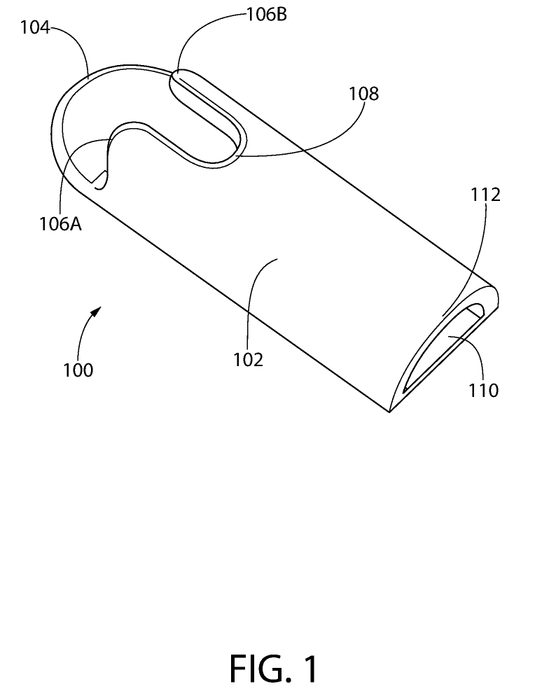

Referring now to FIG. 1, there is shown a close-up view of an exemplary embodiment of a distal end of one of the lumens of the split tip catheter of the present invention. In various embodiments, the catheter assembly 100 comprises a dual lumen having a first lumen and a second lumen that are substantially equal in size, dimension, and shape. Each of the lumens 102 comprises a substantially D-shaped cross section having a flat side 110 and a rounded side 112. The flat sides 110 of each of the lumens 102 can directly face each other such that the rounded sides 112 of the lumens 102 face away from each other.

The distal end of the rounded side 112 comprises a centrally located U-shaped cutout 108 or a U-shaped cutout 108 at a substantial midpoint thereof, defining terminal ends 106A, 106B on either side of the U-shaped cutout 108. The terminal ends 106A, 106B can be rounded or curved and can extend slightly upward toward the distal end 104 of the flat side 110 of the lumen 102 so as to define pockets or grooves where the rounded side 112 and the flat side 110 meet. The U-shaped cutout 108 extends toward the proximal end of the lumen 102 such that the cutout 108 does not extend the entire length of the lumen 102. In the illustrated embodiment, the distal end 104 of the flat side 110 of the lumen 102 extends beyond the terminal ends 106A, 106B of the rounded side 112. The distal end 104 of the flat side 110 extends beyond the terminal ends 106A, 106B in order to prevent recirculation of blood during use. In this regard, it is contemplated that the distal end 104 of the flat side 110 need not extend beyond the terminal ends 106A, 106B when the recirculation of blood does not occur or is not likely.

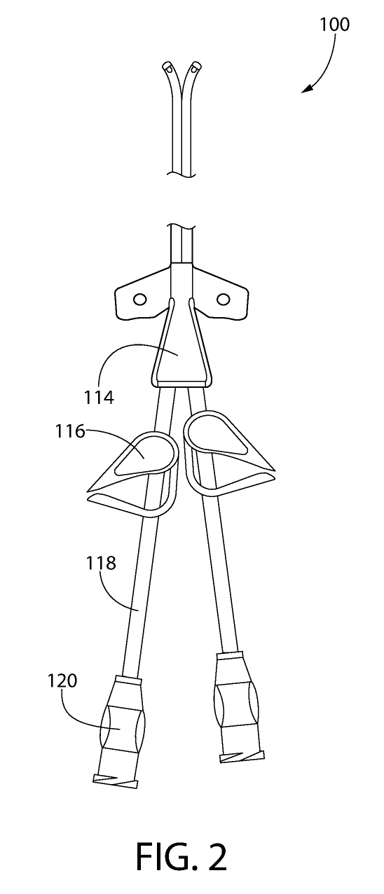

Referring now to FIG. 2, there is shown an overhead view of the split tip catheter 100 of the present invention. The split tip catheter comprises dual lumen with a defined first lumen and a second lumen, each of the lumens comprising a distal end and a proximal end. The first lumen and the second lumen are substantially equal in size, shape, and dimension. In this regard, either of the lumens can be an arterial lumen or a venous lumen and switched readily as needed during use. The dual lumen is formed from a flexible material and may be formed by any suitable method of manufacture, including, but not limited to, injection molding and extrusion processes. In various embodiments, to increase stability and rigidity, the lumens may include any suitable reinforcing material.

The dual lumen is substantially unitary in structure such that the first and second lumens are bonded together from the proximal ends to short of the distal ends where the lumens are split or apart. In this regard, the septum of the dual lumen is split at the distal end, thereby defining the flat sides of each of the first and second lumens. The distal ends of the first and second lumens include a pre-curved configuration in a normal state thereof, wherein the lumens form a substantial Y-shape when viewed from the top in the absence of any external forces. Alternatively, the distal ends of the lumens may be devoid of any such normally curved configuration.

The proximal ends of the lumens are aligned together and connected to a hub 114. The hub 114 may comprise suture holes on opposing sides thereof. In some embodiments, the proximal ends can be reinforced in position via a compression cap or a sleeve. The proximal end of the hub 114 comprises a first and second luer extensions 118 extending therefrom. The first luer extension is in fluid communication with the first lumen and the second luer extension is in fluid communication with the second lumen. In this way, it is contemplated that the hub 114 comprises interior tunnels or openings for facilitating fluid communication between the lumens and the luer extensions 118.

In various embodiments, the luer extensions 118 can include clamps 116 removably attached thereto. The luer extensions 118 can further comprise labels thereon, wherein the labels can be printed or removably affixed to the luer extensions 118. In various embodiments, the clamps 116 can comprise a slide clamp, a pinch clamp, and/or so forth. In the illustrated embodiment, the clamps 116 comprise a pinch clamp. Additionally, the proximal ends of the luer extensions 118 comprise a luer 120. The luer 120 can comprise a female luer, wherein the female luer can connect to a male luer fitted with a spin lock connector.

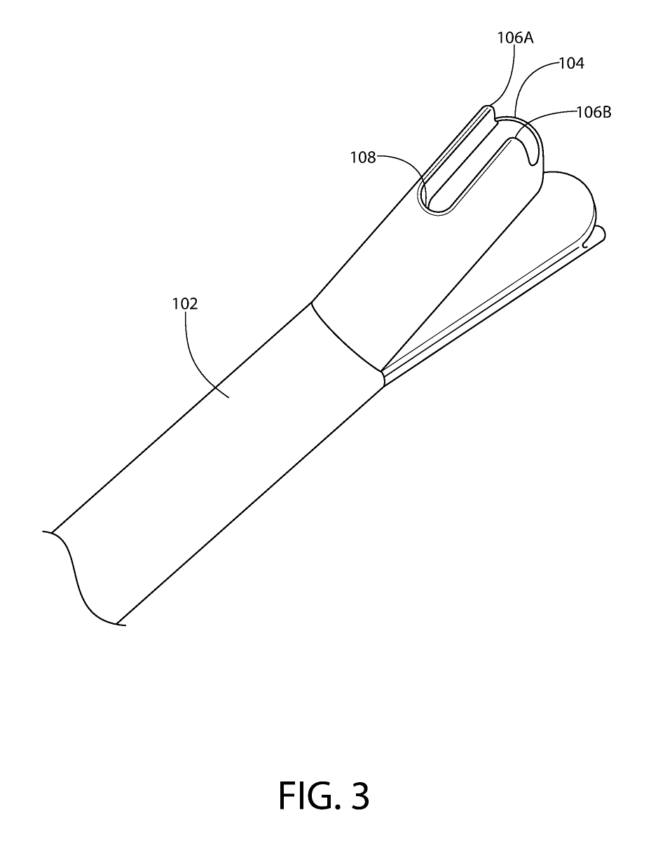

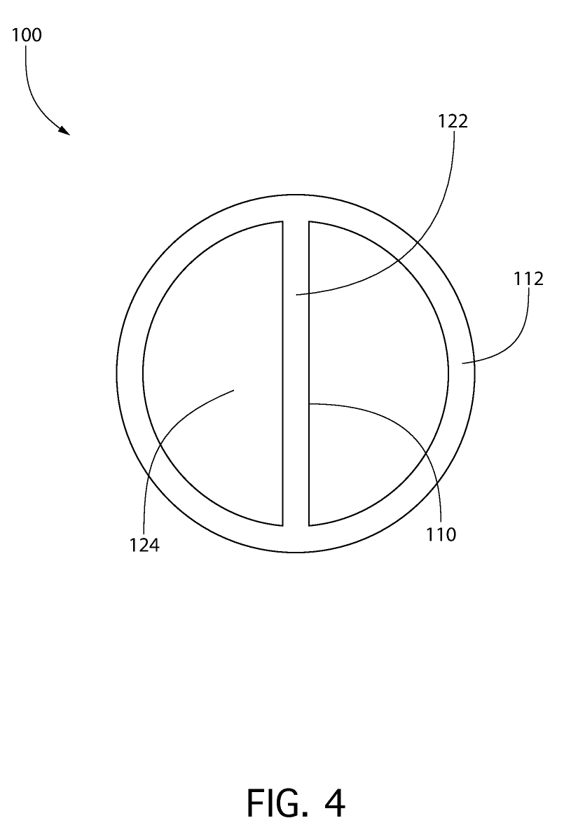

Referring now to FIGS. 3 and 4, there is shown a close-up view of the distal end of the split tip catheter 100 and a cross sectional view of the proximal end of the split tip catheter 100, respectively. The split tip catheter comprises a dual lumen having a substantially circular cross section with a defined first lumen and a second lumen, each of the lumens 102 comprising a distal end and a proximal end. The lumens are separated via a septum 122 that extends between the distal end and the proximal end of each respective lumens, defining an open semicircular sections 124 with an interior volume for each lumen. The septum 122 of the dual lumen can be split at the distal end thereof in order to form a split tip catheter 100.

As noted above, the cross section of each of the lumens at the distal end of the dual lumen is substantially D-shaped. The flat sides 110 of the lumens formed after splitting the septum 122 face toward each other and the rounded sides 112 of the lumens face away from each other to form a substantially round cross section with a septum extending the substantial midpoint thereof.

The distal ends of the lumens are biased outward or bent or angled away from each other such that the catheter 100 resembles a Y-shape when viewed from the top. The angled configuration of the distal ends prevents cleansed blood returning to the body from mixing with the blood going out of the body. It is noted that because the lumens are substantially equal in size, shape, and dimension, the entire catheter assembly is substantially symmetrical. In this way, the lumens are not specifically designated a venous or arterial lumen, the two lumens can be used interchangeably. This is highly advantageous because the connection can be reversed without high recirculation if one of the lumens is blocked or unusable.

Each of the distal ends of the lumens comprises a U-shaped cutout 108 or an opening disposed on a rounded side 112 thereof, and not on the flat side 110 where the lumens join. Additionally, the U-shaped cutout 108 is substantially centered on the rounded side at the distal ends of the lumens, defining terminal ends 106A, 106B. Preferably, the cutouts 108 extend toward the proximal ends of the lumens so that if the distal ends are against the blood vessels, the distal ends do not block the intake of blood during use.

In the illustrated embodiment, the distal end 104 of the flat side 110 does not extend beyond the terminal ends 106A, 106B of the rounded side 112. Preferably, the distal end 104 of the flat side 110 is substantially level with the terminal ends 106A, 106B of the rounded side 112 when no blood recirculation is achieved. Thus, the height of the distal end 104 of the flat side 110 is substantially equal to the height of the terminal ends 106A, 106B of the rounded side 112. In this way, the length of the septum of the lumens can vary depending upon embodiments. More specifically, the length of the septum can be varied depending on blood recirculation problem.

In various embodiments, the present split tip catheter can be formed using a die assembly comprising a heated die. The die comprises a tubular structure with a circular cross section, the cross section comprising a septum disposed at a substantial midsection thereof so as to split the cross section (i.e., the inside) of the die into two semicircle sections. The die is configured to receive a pair of mandrels therethrough, wherein the mandrels comprise a D-shaped cross section. More specifically, each of the mandrels is configured to be inserted through a semicircle section of the die. Additionally, the mandrels are configured to support the dual lumen thereon. As discussed above, the dual lumen comprises a circular cross section with a septum extending therethrough, defining two open semicircle sections, similar to the die. The septum of the die is aligned with the septum of the dual lumen such that the septum of the die is configured to split the dual lumen into the first lumen and the second lumen.

The die is heated to a desired temperature via a heating unit. In a preferred embodiment, the die is heated to 300° F. to 350° F., via, for example, a heating unit utilizing radio frequency technology. The mandrels are positioned within the semicircle sections of the dual lumen (e.g., having an inside diameter of 0.060″×0.130″ according to one embodiment) and then the dual lumen, while loaded on the mandrels, are inserted into the die. It is noted that the mandrels help prevent the dual lumen from collapsing when heat is applied.

The dual lumen and the mandrels are pushed, for example, via a block, into the die until the dual lumen passes through the septum within the die and two lumens are fully formed. The combination of properly guiding the dual lumen and the heat creates a smooth finish. In this regard, no additional process is needed to improve the finish of the split lumens. The separated lumens are cooled down with air blast via a cooling unit. The lumens are removed from the mandrels. In this regard, regular dual lumens can be used and the septum of the dual lumen do not need to have additional thickness.

It is therefore submitted that the instant invention has been shown and described in what is considered to be the most practical and preferred embodiments. It is recognized, however, that departures may be made within the scope of the invention and those obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

1. A split tip catheter assembly, comprising:
a first lumen and a second lumen, each of the first lumen and the second lumen having a proximal end and a distal end, wherein the first lumen and the second lumen are bonded at the proximal end and separated at the distal end;
the distal end of the first lumen and the second lumen bent away from each other; and
a cutout disposed on the distal end of each of the first lumen and the second lumen.

2. The split tip catheter assembly of claim 1, wherein a cross section of each of the first lumen and the second lumen is D-shaped.

3. The split tip catheter assembly of claim 1, wherein the cutout is U-shaped.

4. The split tip catheter assembly of claim 1, wherein each of the first lumen and the second lumen comprises a flat side and a rounded side, further wherein the cutout is disposed on the rounded side of the first lumen and the second lumen.

5. The split tip catheter assembly of claim 1, wherein the first lumen and the second lumen are substantially symmetrical.

6. The split tip catheter assembly of claim 1, wherein a cross section of the proximal end of the first lumen and the second lumen is substantially circular.

7. The split tip catheter assembly of claim 1, wherein the proximal end of each of the first lumen and the second lumen is connected to a hub that is in fluid communication with luer extensions.

8. A split tip catheter assembly, comprising:
a dual lumen having a proximal end and a distal end and a septum that extends between the proximal end and the distal end, wherein the septum is split at the distal end to define a first lumen and a second lumen having a flat side and a rounded side; and
a U-shaped cutout disposed on the rounded side of each of the first lumen and the second lumen at the distal end of the dual lumen.

9. The split tip catheter assembly of claim 8, wherein the flat side of the first lumen and the second lumen face toward each other and the rounded side of the first lumen and the second lumen face away from each other.

10. The split tip catheter assembly of claim 8, wherein the U-shaped cutout on the rounded side defines terminal ends on either side of the U-shaped cutout.

11. The split tip catheter assembly of claim 10, wherein the terminal ends are substantially level with the distal end of the flat side.

12. The split tip catheter assembly of claim 10, wherein the distal end of the flat side extends beyond the terminal ends.

13. The split tip catheter assembly of claim 8, wherein the proximal end of the dual lumen is connected to a hub that is in fluid communication with luer extensions.

14. The split tip catheter assembly of claim 8, wherein the first lumen and the second lumen are bent away from each other to form a substantial Y-shape when viewed from the top.

15. A split tip catheter assembly, comprising:
a dual lumen having a proximal end and a distal end and a septum that extends between the proximal end and the distal end, wherein the septum is split at the distal end to define a first lumen and a second lumen having a flat side and a rounded side, the flat side being defined by the split portion of the septum;
a U-shaped cutout disposed on the rounded side of each of the first lumen and the second lumen at the distal end of the dual lumen, wherein the U-shaped cutout is centrally located on the rounded side to define terminal ends on either side of the U-shaped cutout; and
a hub connected to the proximal end of the dual lumen.

16. The split tip catheter assembly of claim 15, wherein the terminal ends are substantially level with the distal end of the flat side.

17. The split tip catheter assembly of claim 15, wherein the distal end of the flat side extends beyond the terminal ends.

18. The split tip catheter assembly of claim 15, wherein the first lumen and the second lumen are bent away from each other to form a substantial Y-shape when viewed from the top.

19. The split tip catheter assembly of claim 15, further comprising luer extensions attached to the hub, the luer extensions in fluid communication with the dual lumen.

20. The split tip catheter assembly of claim 15, wherein the terminal ends are rounded and extend upward toward the distal end of the flat side to define grooves where the rounded side and the flat side meet.
